(19) United States
(12) Patent Application Publication
(10) Pub. No.: US 2023/0293676 A1
(43) Pub. Date: Sep. 21, 2023

(54) Pharmaceutical Composition Comprising Hepatitis B Virus-Derived Polypeptide for Prevention or Treatment of Cancer

(71) Applicant: SEOUL NATIONAL UNIVERSITY R&DB FOUNDATION, Seoul (KR)

(72) Inventors: Bum Joon Kim, Seoul (KR); Su Bin Yang, Seoul (KR); Mi Hyun Lee, Seoul (KR)

(73) Assignee: SEOUL NATIONAL UNIVERSITY R&DB FOUNDATION, Seoul (KR)

(21) Appl. No.: 18/021,262

(22) PCT Filed: Aug. 12, 2021
(86) PCT No.: PCT/KR2021/010710

§ 371 Date: Feb. 14, 2023

(30) Foreign Application Priority Data
Aug. 14, 2020 (KR) 10-2020-0102774

(51) Int. Cl. A61K 39/29; A61K 39/395; A61P 35/00

(52) U.S. Cl. CPC A61K 39/292; A61K 39/3955; A61P 35/00; A61K 2039/585

(57) ABSTRACT

One aspect relates to a pharmaceutical composition for the prevention or treatment of cancer or an anticancer immune vaccine composition, including a polypeptide consisting of the amino acid sequence of SEQ ID NO: 1. The composition can enhance anticancer immunity by activating dendritic cells and T cells, and furthermore, can exhibit a remarkably excellent synergistic anticancer immune effect through administration in combination with an immune checkpoint inhibitor.

### TECHNICAL FIELD

The present disclosure relates to a pharmaceutical composition for the prevention or treatment of cancer, including a hepatitis B virus-derived polypeptide.

### BACKGROUND ART

Cancer is a disease caused by continuous division due to mutations in genes that regulate the cell cycle in normal cells. This means that humans have genes that can cause cancer from birth, and about 60 trillion cells in the body can develop into cancer cells.

Unlike benign tumors, cancer cells have a fast growth rate and can divide and proliferate indefinitely. In addition, cancer cells can infiltrate into the surrounding tissues and can migrate throughout the body via blood or lymph fluid. This enables metastasis in which cancer cells are found not only in one tissue but also in other tissues.

Unlike infectious diseases, cancer, which is a malignant tumor, does not occur only in a specific region, but occurs worldwide. According to the WHO report, about 14 million patients were newly diagnosed with cancer in 2012 alone worldwide, about 33 million patients were suffering from cancer, and about 8.2 million patients died of cancer that year.

Although the normal immune function of the human body has the ability to destroy up to 10 million tumor cells generated in the body, an excessive number of cancer cells proliferated is insufficient to be eliminated by the function of immune cells. For this reason, there is a limitation on eliminating cancer cells with only a normal immune response.

Surgical resection is primarily required to treat cancer, but there is a limitation in that surgical operation is restricted depending on the size of cancer and the degree of metastasis.

Chemotherapy, which is one of the most widely used anticancer treatments, has a limitation in that, since a specific therapeutic agent is applicable only to specific cancer, the development of therapeutic agents for various cancers is required. In addition, since cancer cells originate from normal cells, chemotherapy also acts on normal cells when applied, thus causing 19 types of side effects.

Considering that cancer mainly occurs in elderly patients, side effects caused by the above therapy are a considerable burden to patients, and the prolongation of life through treatment may not be considered proportional to the improvement of quality of life.

Thus, tumor immunotherapy that can be applied to various cancers and has fewer side effects is attracting great attention.

Unlike conventional anticancer drugs that attack cancer cells themselves, cancer immunotherapeutic drugs are therapeutic agents that stimulate the immune system to induce immune cells to selectively attack only cancer cells. These cancer immunotherapeutic drugs include immune checkpoint inhibitors (CTLA4 inhibitor and PD-L1 inhibitor), immune cell therapeutic agents, and the like.

Immunotherapeutic drugs have a mechanism to kill cancer cells by activating immune cells of the human body. These immunotherapeutic drugs can be used in various types of cancer even without a specific genetic mutation. In particular, cancer immunotherapeutic drugs have fewer side effects in terms of treating cancer by enhancing patient's own immune capability, and have the effects of improving the quality of life of cancer patients and remarkably extending the survival period.

Therefore, on the basis of the fact that poly6 peptide derived from Hepatitis B virus activates dendritic cells and induces the ability to differentiate into Tip-DCs, poly6 is intended to be used in the development of novel anticancer vaccines.

### DETAILED DESCRIPTION OF THE DISCLOSURE

### Technical Problem

One aspect is to provide a pharmaceutical composition for the prevention or treatment of cancer, including a polypeptide including the amino acid sequence of SEQ ID NO: 1.

Another aspect is to provide an anticancer immune vaccine composition including a polypeptide including the amino acid sequence of SEQ ID NO: 1.

Another aspect is to provide a composition for enhancing anticancer immunity, including a polypeptide including the amino acid sequence of SEQ ID NO: 1.

Another aspect is to provide an anticancer immune adjuvant including a polypeptide including the amino acid sequence of SEQ ID NO: 1.

Another aspect is to provide a health functional food for enhancing anticancer immunity, including a polypeptide including the amino acid sequence of SEQ ID NO: 1.

Another aspect is to provide a cancer immunotherapy method including administering a polypeptide including the amino acid sequence of SEQ ID NO: 1 to a subject in need thereof.

Another aspect is to provide a method for enhancing anticancer immunity, including administering a polypeptide including the amino acid sequence of SEQ ID NO: 1 to a subject in need thereof.

### Technical Solution

One aspect provides a pharmaceutical composition for the prevention or treatment of cancer, including a polypeptide consisting of the amino acid sequence of SEQ ID NO: 1.

The term “polypeptide” refers to a polymer consisting of two or more amino acids linked by amide bonds (or peptide bonds). The polypeptide may consist of the amino acid sequence of SEQ ID NO: 1. The polypeptide may include a polypeptide having at least about 70%, at least about 75%, at least about 80%, at least about 85%, at least about 90%, at least about 92%, at least about 95%, at least about 97%, at least about 98%, or at least about 99% sequence homology to the amino acid sequence of SEQ ID NO: 1.

In the present specification, the peptide or amino acids thereof may be substituted conservatively or non-conservatively.

The term “conservative substitution” as used herein refers to the replacement of an amino acid present in the natural nucleotide sequence in the peptide with a naturally or non-naturally occurring amino acid or a peptidomimetics having similar steric properties. When the side chain of the naturally occurring amino acid to be substituted is polar or hydrophobic, the conservative substitution should be made with a naturally occurring amino acid, non-naturally occurring amino acid or peptidomimetic moiety that is polar or hydrophobic (other than having the same steric properties as the side chain of the substituted amino acid).

Naturally occurring amino acids are typically classified according to the properties thereof, and thus, conservative substitutions by naturally occurring amino acids can be easily determined in consideration of the fact that charged amino acids according to the present disclosure are substituted with sterically similar uncharged amino acids, which are considered conservative substituents.

Amino acid analogs known in the art (synthetic amino acids) may also be used to make conservative substitutions with non-naturally occurring amino acids. Peptidomimetics of naturally occurring amino acids are well documented in the literature known to those of ordinary skill in the art.

When a conservative substitution is made, the substituted amino acid must have the same or similar functional groups on the side chain as the original amino acid.

The term “non-conservative substitutions” as used herein refers to the replacement of an amino acid as present in the parent sequence by another naturally or non-naturally occurring amino acid having different electrochemical and/or steric properties. Thus, the side chain of the substituted amino acid may be significantly larger than the side chain of a native amino acid to be substituted and/or may have functional groups with electrical properties that differ significantly from those of the substituted amino acid. Specific examples of these non-conservative substituents include substituents such as phenylalanine or cyclohexylmethylglycine for alanine, isoleucine for glycine, or —NH—CH[(—CH₂)₅—COOH]—CO— for aspartic acid.

Although the peptide or polypeptide of the present specification is used in a linear form, it will be appreciated that, unless cyclization severely interferes with peptide characteristics, cyclic forms of the peptide may also be used.

Since the peptide or polypeptide of the present specification is used in therapeutic agents that require the same to be present in soluble form, the peptide or polypeptide of some embodiments of the present specification includes one or more non-natural or natural polar amino acids, i.e., serine and threonine which are capable of increasing the stability of the peptide or the polypeptide due to the hydroxyl-containing side chains thereof.

The N-terminus and C-terminus of the peptide or polypeptide of the present specification may be protected by functional groups. Suitable functional groups are described in Green and Wuts, “Protecting Groups in Organic Synthesis”, John Wiley and Sons, Chapters 5 and 7, 1991, the contents of which are incorporated herein by reference. Thus, the peptide or polypeptide may be modified at the N-(amine) terminus and/or the C-(carboxyl) terminus thereof to produce an end-capping modified peptide.

The phrases “end-capping modified polypeptide” and “protected polypeptide” as used herein are interchangeably used herein, and refer to a polypeptide which has been modified at the N-(amine) terminus and/or the C-(carbonyl) terminus thereof. The end-capping modification refers to the attachment of a chemical moiety to the terminus of the polypeptide, to form a cap. Such a chemical moiety is referred to herein as an end-capping moiety and is typically also referred to, herein and in the art, interchangeably as a peptide protecting moiety or a functional group. Hydroxyl protecting groups include, but are not limited to, esters, carbonates, and carbamate protecting groups. Amine protecting groups include, but are not limited to, alkoxy and aryloxy carbonyl groups. Carboxylic acid protecting groups include, but are not limited to, aliphatic esters, benzylic esters, and aryl esters.

The phrase “end-capping moiety” as used herein refers to a moiety that, when attached to the terminus, modifies the N-terminus and/or C-terminus of the peptide. The end-capping modification typically results in masking the charge of the peptide terminus, and/or altering chemical properties thereof, such as hydrophobicity, hydrophilicity, reactivity, and solubility. By selecting the nature of the end-capping modification, the hydrophobicity/hydrophilicity, as well as the solubility of the peptide can be finely controlled. According to specific embodiments, the protecting groups facilitate transport of the peptide attached thereto into a cell. These moieties can be cleaved in vivo, either by hydrolysis or enzymatically, inside the cell.

According to specific embodiments, the end-capping includes N-terminus end-capping. Representative examples of N-terminus-capping moieties include, but are not limited to, formyl, acetyl (also referred to herein as “AC”), trifluoroacetyl, benzyl, benzyloxycarbonyl (also referred to herein as “Cbz”), tert-butoxycarbonyl (also referred to herein as “Boc”), trimethylsilyl (also referred to herein as “TMS”), 2-trimethylsilyl-ethanesulfonyl (also referred to herein as “SES”), trityl and the substituted trityl groups, allyloxycarbonyl, 9-fluorenylmethyloxycarbonyl (also referred to herein as “Fmoc”), and nitro-veratryloxycarbonyl (“NVOC”).

According to specific embodiments, the end-capping includes C-terminus end-capping. Examples of C-terminus end-capping moieties are typical moieties that induce acylation of the carboxyl group at the C-terminus and may include benzyl and trityl ethers as well as alkyl ethers, tetrahydropyranyl ethers, trialkylsilyl ethers, allyl ethers, monomethoxytrityl, and dimethoxytrityl. Alternatively, the -COON group of the C-terminus end-capping may be modified to an amide group.

Other end-capping modifications of peptides include replacement of the amine and/or carboxyl with a different moiety, such as hydroxyl, thiol, halide, alkyl, aryl, alkoxy, and aryloxy.

Furthermore, the polypeptide may additionally include an amino acid sequence which is prepared for the specific purpose of a targeting sequence, a tag, or a labeled residue.

The term “homology” is intended to refer to the degree of similarity to a wild-type amino acid sequence, and comparison in the homology may be performed using a comparison program widely known in the art, and homology between two or more sequences may be calculated as a percentage (%).

The polypeptide may be derived from a natural source, or may be obtained by various polypeptide synthesis methods widely known in the art. For example, the polypeptide may be prepared by polynucleotide recombination and protein expression systems, or by in-vitro synthesis through chemical synthesis such as peptide synthesis, and by cell-free protein synthesis. In addition, for example, the polypeptide may be a peptide, an extract of plant-derived tissues or cells, or a product obtained by culturing microorganisms (e.g., bacteria or fungi, particularly, yeasts), and particularly, the polypeptide may be derived from hepatitis B virus (HBV) polymerase, and more particularly, may be derived from the preS1 region of the HBV polymerase.

In one aspect, the polypeptide may activate dendritic cells and T cells, and particularly, may increase the expression of at least one selected from the group consisting of TNF-a and iNOS in dendritic cells, and thus may exhibit an anticancer immune effect.

In addition, the polypeptide may increase the expression of at least one selected from the group consisting of CD80, CD86, and MHCI in dendritic cells.

The term “cancer” refers to a class of diseases characterized by the development of abnormal cells that proliferate uncontrollably and have the ability to invade and destroy normal body tissue.

The cancer may be at least one cancer selected from the group consisting of acoustic neuroma, adenocarcinoma, adrenal gland cancer, anal cancer, angiosarcoma (e.g., lymphangiosarcoma), lymphangioendotheliosarcoma, hemangiosarcoma, appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, and medullary carcinoma of the breast), brain cancer (e.g., meningioma and glioblastoma), glioblastoma (e.g., astrocytoma, oligodendroglioma, and medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, and colorectal adenocarcinoma), connective tissue cancer, epithelial carcinoma, ependymoma, endotheliosarcoma (e.g., Kaposi's sarcoma and multiple idiopathic hemorrhagic sarcoma), endometrial cancer (e.g., uterine cancer and uterine sarcoma), esophageal cancer (e.g., adenocarcinoma of the esophagus and Barrett's adenocarcinoma), Ewing's sarcoma, ocular cancer (e.g., intraocular melanoma, and retinoblastoma), familiar hypereosinophilia, gall bladder cancer, gastric cancer (e.g., stomach adenocarcinoma), gastrointestinal stromal tumor (GIST), germ cell cancer, head and neck cancer (e.g., head and neck squamous cell carcinoma), oral cancer (e.g., oral squamous cell carcinoma), throat cancer (e.g., laryngeal cancer, pharyngeal cancer, nasopharyngeal cancer, and oropharyngeal cancer), heavy chain disease (e.g., alpha chain disease, gamma chain disease, and mu chain disease), hemangioblastoma, hypopharynx cancer, inflammatory myofibroblastic tumors, immunocytic amyloidosis, kidney cancer (e.g., nephroblastoma, also known as Wilms' tumor, and renal cell carcinoma), liver cancer (e.g., hepatocellular cancer (HCC) and malignant hepatoma), lung cancer (e.g., bronchogenic carcinoma, small cell lung cancer (SCLC), non-small cell lung cancer (NSCLC), and adenocarcinoma of the lung), leiomyosarcoma (LMS), mastocytosis (e.g., systemic mastocytosis), muscle cancer, myelodysplastic syndrome (MDS), mesothelioma, myeloproliferative disorder (MPD) (e.g., polycythemia Vera (PV), essential thrombocytosis (ET), unidentified agnogenic myeloid metaplasia (AMM) also known as myelofibrosis (MF), chronic idiopathic myelofibrosis, chronic myelocytic leukemia (CML), chronic neutrophilic leukemia (CNL), and hypereosinophilic syndrome (HES)), neuroblastoma, neurofibroma (e.g., neurofibromatosis (NF) type 1 or type 2, and schwannomatosis), neuroendocrine cancer (e.g., gastroenteropancreatic neuroendocrine tumor (GEP-NET) and carcinoid tumor), osteosarcoma (e.g., bone cancer), ovarian cancer (e.g., cystadenocarcinoma, ovarian embryonal carcinoma, and ovarian adenocarcinoma), papillary adenocarcinoma, pancreatic cancer (e.g., pancreatic andenocarcinoma, intraductal papillary mucinous neoplasm (IPMN), and Islet cell tumors), penile cancer (e.g., Paget's disease of the penis and scrotum), pinealoma, primitive neuroectodermal tumor (PNT), plasma cell neoplasia, paraneoplastic syndrome, intraepithelial neoplasm, prostate cancer (e.g., prostate adenocarcinoma), rectal cancer, rhabdomyosarcoma, salivary gland cancer, skin cancer (e.g., squamous cell carcinoma (SCC)), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, and myxosarcoma), sebaceous gland carcinoma, small intestine cancer, sweat gland carcinoma, synovioma, testicular cancer (e.g., seminoma and testicular embryonal carcinoma), thyroid cancer (e.g., papillary carcinoma of the thyroid, papillary thyroid carcinoma (PTC), and medullary thyroid cancer), urethral cancer, vaginal cancer, and vulvar cancer (e.g., Paget's disease of the vulva), and particularly, may be at least one cancer selected from the group consisting of colon cancer and melanoma.

The term “prevention” may refer to all actions that inhibit cancer of a subject or delay the onset thereof via administration of a pharmaceutical composition according to an aspect.

The term “treatment” may refer to all actions that alleviate or beneficially change symptoms of cancer of a subject via administration of a pharmaceutical composition according to an aspect.

The pharmaceutical composition may include the active ingredient alone or may be provided as a pharmaceutical composition including one or more pharmaceutically acceptable carriers, excipients or diluents.

Specifically, the carrier may be, for example, a colloidal suspension, powder, saline, lipid, liposomes, microspheres, or nanospheric particles. These may form a complex with a carrier or may be associated with the carrier, and may be delivered in vivo using a delivery system which is known in the art, such as lipids, liposomes, microparticles, gold, nanoparticles, polymers, condensation reagents, polysaccharides, polyamino acids, dendrimers, saponins, adsorption enhancers, or fatty acids.

When the pharmaceutical composition is formulated, the pharmaceutical composition may be prepared using commonly used diluents or excipients, such as lubricants, sweeteners, flavoring agents, emulsifiers, suspensions, preservatives, fillers, extenders, binders, wetting agents, disintegrants, and surfactants. Solid preparations for oral administration may include tablets, pills, powders, granules, capsules, and the like, and such a solid formulation may be prepared by mixing the composition with at least one excipient, for example, starch, calcium carbonate, sucrose, lactose, and gelatin. In addition to simple excipients, lubricants such as magnesium stearate and talc may also be used. Liquid preparations for oral administration may include suspension, solutions for internal use, emulsions, syrups, and the like. In addition to water and liquid paraffin, which are commonly used simple diluents, various excipients, for example, wetting agents, sweeteners, fragrances, and preservatives may be included. Preparations for parenteral administration may include sterile aqueous solutions, non-aqueous solvents, suspensions, emulsions, lyophilized preparations, and suppositories. For the non-aqueous solvents and the suspensions, propylene glycol, polyethylene glycol, vegetable oils such as olive oil, injectable ester such as ethyl oleate, and the like may be used. As bases of the suppositories, Witepsol, macrogol, Tween 61, cacao fat, laurin fat, glycerogeratin, and the like may be used. When prepared in the form of eye drops, a known diluent, excipient, or the like may be used.

In addition, in one aspect, the pharmaceutical composition may further include an immune checkpoint inhibitor.

The immune checkpoint inhibitor may be an existing immune checkpoint inhibitor or a newly developed immune checkpoint inhibitor. When the pharmaceutical composition further includes an immune checkpoint inhibitor, it is important to mix the immune checkpoint inhibitor in an amount sufficient to obtain the maximum effect with the minimum amount without side effects, and the amount may be easily determined by those of ordinary skill in the art.

The immune checkpoint inhibitor may be, for example, at least one selected from the group consisting of a programmed cell death ligand-1 (PD-L1) inhibitor and a cytotoxic T-lymphocyte-associated protein 4 (CTLA4) inhibitor, and particularly, may be PD-L1.

When the pharmaceutical composition further includes an immune checkpoint inhibitor, a more pronounced synergistic anticancer effect, i.e., more pronounced effects on tumor growth inhibition, immune activity and the like, may be exhibited compared to when the pharmaceutical composition includes the polypeptide alone as the active ingredient.

In addition, in one aspect, the pharmaceutical composition may be administered in combination with an immune checkpoint inhibitor for single administration.

The immune checkpoint inhibitor may be an existing immune checkpoint inhibitor or a newly developed immune checkpoint inhibitor, and the pharmaceutical composition may be administered in combination with the immune checkpoint inhibitor, administered simultaneously, separately, or sequentially, and administered in a single dose or in multiple doses. It is important to administer in an amount sufficient to obtain the maximum effect with the minimum amount without side effects, considering the factors described above, and the amount may be easily determined by those of ordinary skill in the art.

The immune checkpoint inhibitor may be, for example, at least one selected from the group consisting of a programmed cell death ligand-1 (PD-L1) inhibitor and a cytotoxic T-lymphocyte-associated protein 4 (CTLA4) inhibitor, and particularly, may be PD-L1.

When the pharmaceutical composition is administered in combination with an immune checkpoint inhibitor, a more pronounced synergistic anticancer effect, i.e., more pronounced effects on tumor growth inhibition, immune activity and the like, may be exhibited compared to when the pharmaceutical composition is administered alone.

The term “administration” refers to providing a subject with a predetermined substance by using an appropriate method, and the term “subject” refers to all organisms such as rats, mice, and livestock, including humans which can have cancer. As a specific example, the subject may be mammals including humans.

The composition may be administered orally or parenterally, and parenteral administration may be performed using a method selected from external application to the skin or intraperitoneal injection, intrarectal injection, subcutaneous injection, intravenous injection, intramuscular injection, intraarterial injection, intramedullary injection, intracardiac injection, intrathecal injection, transdermal injection, intranasal injection, intra-intestinal injection, local injection, sublingual injection, intrarectal injection, or intrathoracic injection.

The pharmaceutical composition is administered in a pharmaceutically effective amount. The term “pharmaceutically effective amount” refers to an amount sufficient to treat diseases at a reasonable benefit/risk ratio applicable to medical treatment, and an effective dosage level may be determined according to factors including type of diseases of patients, the severity of disease, the activity of drugs, sensitivity to drugs, administration time, administration route, excretion rate, treatment period, and simultaneously used drugs, and other factors well known in the medical field. Specifically, the pharmaceutical composition may be administered at a dose of 0.001 mg/kg/day to 1000 mg/kg/day, and more particularly, 0.1 mg/kg/day to 100 mg/kg/day. The composition may be administered once a day or may also be administered in multiple doses.

In one aspect, the pharmaceutical composition may be administered once a day or in multiple doses. For example, the pharmaceutical composition may be administered every other day or one day a week.

Specifically, the effective amount of the pharmaceutical composition may vary according to the age, gender, condition, and body weight of a patient, the absorption, inactivity, and excretion rate of active ingredients in the body, the type of disease, and simultaneously used drugs, and may be increased or decreased according to administration route, the severity of obesity, gender, body weight, age, and the like.

Another aspect provides an anticancer immune vaccine composition including a polypeptide consisting of the amino acid sequence of SEQ ID NO: 1.

The term “vaccine” refers to a pharmaceutical composition containing at least one immunologically active component that induces an immunological response in an animal. The immunologically active component of a vaccine may contain appropriate elements of live or dead viruses or bacteria (subunit vaccines), whereby these elements are produced by destroying the whole viruses or bacteria or the growth cultures thereof, and subsequent purification steps yielding the desired structure(s), or by synthetic processes induced by an appropriate manipulation of a suitable system based on bacteria, insects, mammals or other species, followed by isolation and purification processes, or by induction of the synthetic processes in the animal in need of a vaccine by direct incorporation of a genetic material using suitable pharmaceutical compositions (polynucleotide vaccination). A vaccine may include one or simultaneously one or more of the elements described above.

In one aspect, the polypeptide may activate dendritic cells and T cells, and specifically, may increase the expression of at least one selected from the group consisting of TNF-α and iNOS in dendritic cells, and thus may exhibit an anticancer immune effect.

In addition, the polypeptide may increase the expression of at least one selected from the group consisting of CD40, CD80, CD86 and MHCII in dendritic cells.

In one aspect, the cancer may be at least one selected from the group consisting of colon cancer and melanoma.

In addition, in one aspect, the vaccine composition may further include an immune checkpoint inhibitor.

The immune checkpoint inhibitor may be an existing immune checkpoint inhibitor or a newly developed immune checkpoint inhibitor. When the pharmaceutical composition further includes an immune checkpoint inhibitor, it is important to mix the immune checkpoint inhibitor in an amount sufficient to obtain the maximum effect with the minimum amount without side effects, and the amount may be easily determined by those of ordinary skill in the art.

The immune checkpoint inhibitor may be, for example, at least one selected from the group consisting of a programmed cell death ligand-1 (PD-L1) inhibitor and a cytotoxic T-lymphocyte-associated protein 4 (CTLA4) inhibitor, and particularly, may be PD-L1.

When the vaccine composition further includes an immune checkpoint inhibitor, a more pronounced synergistic anticancer effect, i.e., more pronounced effects on tumor growth inhibition, immune activity and the like, may be exhibited compared to when the vaccine composition includes the polypeptide alone as an active ingredient.

In addition, in one aspect, the vaccine composition may be administered in combination with an immune checkpoint inhibitor for single administration.

The immune checkpoint inhibitor may be an existing immune checkpoint inhibitor or a newly developed immune checkpoint inhibitor, and the vaccine composition may be administered in combination with the immune checkpoint inhibitor, administered simultaneously, separately, or sequentially, and administered in a single dose or in multiple doses. It is important to administer in an amount sufficient to obtain the maximum effect with the minimum amount without side effects considering the factors described above, and the amount may be easily determined by those of ordinary skill in the art.

The immune checkpoint inhibitor may be, for example, at least one selected from the group consisting of a programmed cell death ligand-1 (PD-L1) inhibitor and a cytotoxic T-lymphocyte-associated protein 4 (CTLA4) inhibitor, and particularly, may be PD-L1.

When the pharmaceutical composition is administered in combination with an immune checkpoint inhibitor, a more pronounced synergistic anticancer effect, i.e., more pronounced effects on tumor growth inhibition, immune activity and the like, may be exhibited compared to when the pharmaceutical composition is administered alone.

The vaccine composition may be administered to a subject in an immunologically effective amount. The term “immunologically effective amount” refers to an amount sufficient to exhibit an immune activity enhancement effect and an amount sufficient not to cause side effects or serious or excessive immune responses, and the exact administration concentration varies depending on the specific immunogen to be administered, and may be easily determined by those of ordinary skill in the art according to factors well known in the medical field, such as the age, body weight, health, and gender of a subject to be vaccinated, the sensitivity of a subject to a drug, administration route, and administration method.

The vaccine composition according to one aspect may include an immunologically acceptable vaccine protecting agent, an immunity-enhancing agent, a diluent, an absorption promoter, and the like, if necessary. The vaccine protecting agent may include, for example, a lactose phosphate glutamate gelatin mixture. The immunity-enhancing agent may include, for example, aluminum hydroxide, mineral oil or other oils, or auxiliary molecules added to a vaccine or produced by the body after each induction by these additional components, such as interferons, interleukins or growth factors. When the vaccine is a solution or injection, the vaccine may contain propylene glycol and sodium chloride in an amount (e.g., about 1%) sufficient to prevent hemolysis, if necessary.

Another aspect provides a composition for enhancing anticancer immunity, including a polypeptide consisting the amino acid sequence of SEQ ID NO: 1.

The terms “polypetide,” “anticancer immunity,” “composition” and the like may be within the above-described ranges.

Another aspect provides an anticancer immune adjuvant including a polypeptide consisting the amino acid sequence of SEQ ID NO: 1.

The terms “polypeptide,” “anticancer,” “immunity” and the like may be within the above-described ranges, and the immune adjuvant may assist or enhance the immunity-enhancing activity of an existing vaccine.

Another aspect provides a health functional food for enhancing anticancer immunity, including a polypeptide consisting of the amino acid sequence of SEQ ID NO: 1.

The terms “polypeptide,” “cancer,” “immunity” and the like may be within the above-described ranges.

The term “amelioration” may refer to all actions that decrease at least the degree of parameters related to conditions being treated, e.g., symptoms. In this regard, the health functional food may be used before or after the onset of the corresponding disease, simultaneously with or separately from a drug for treatment, to prevent or ameliorate cancer.

In the health functional food, the active ingredient may be added to food as it is or used together with other food or food ingredients, and may be appropriately used according to conventional methods. The mixing amount of the active ingredient may be suitably determined depending on the purpose of use thereof (for prevention or amelioration). In general, when food or beverage is prepared, the health functional food may be added in an amount of about 15 wt % or less, and more particularly, about 10 wt % or less, with respect to the raw material. However, in the case of long-term ingestion for health and hygienic purposes or for health control purposes, the amount may be the above range or less.

The health functional food may be formulated as one selected from the group consisting of tablets, pills, powders, granules, powders, capsules, and liquid formulations by further including at least one of carriers, diluents, excipients, and additives. Examples of foods to which a compound according to one aspect may be added include various foods, powders, granules, tablets, capsules, syrups, beverages, gum, tea, vitamin complexes, health functional foods, and the like.

Specifically, the carriers, excipients, diluents, and additives may be at least one selected from the group consisting of lactose, dextrose, sucrose, sorbitol, mannitol, erythritol, starch, acacia gum, calcium phosphate, alginate, gelatin, calcium phosphate, calcium silicate, microcrystalline cellulose, polyvinylpyrrolidone, cellulose, polyvinylpyrrolidone, methylcellulose, water, sugar syrup, methylcellulose, methyl hydroxy benzoate, propylhydroxy benzoate, talc, magnesium stearate, and mineral oil.

In addition to the active ingredient, the health functional food may contain other ingredients as essential ingredients without particular limitation. For example, the health functional food may contain various flavoring agents or natural carbohydrates as additional ingredients like general beverages. Examples of the above-described natural carbohydrates include:

monosaccharides, e.g., glucose and fructose; disaccharides, e.g., maltose and sucrose; and polysaccharides, e.g., general sugars such as dextrin and cyclodextrin, and sugar alcohols such as xylitol, sorbitol, and erythritol. As a flavoring agent other than the above-described flavoring agents, a natural flavoring agent (thaumatin and stevia extracts (e.g., rebaudioside A, glycyrrhizin, and the like) and a synthetic flavoring agent (saccharin, aspartame, and the like) are preferably used. The proportion of the natural carbohydrates may be appropriately determined by those of ordinary skill in the art via selection.

In addition to the above-listed ingredients, the health functional food according to one aspect may include various nutritional supplements, vitamins, minerals (electrolytes), flavors such as synthetic flavors and natural flavors, colorants and enhancers (cheese, chocolate, and the like), pectic acid and salts thereof, alginic acid and salts thereof, organic acids, a protective colloidal thickener, a pH adjuster, a stabilizer, a preservative, glycerin, alcohols, a carbonating agent used in carbonated beverages, and the like. These ingredients may be used alone or a combination thereof may be used, and the proportion of these additives may also be appropriately selected by those of ordinary skill in the art.

Another aspect provides a method of preventing or treating cancer, including administering a polypeptide consisting of the amino acid sequence of SEQ ID NO: 1 to a subject in need thereof.

Another aspect provides a cancer immunotherapy method including administering a polypeptide consisting of the amino acid sequence of SEQ ID NO: 1 to a subject in need thereof.

The terms “polypeptide,” “subject,” “administration,” “cancer” and the like may be within the above-described ranges.

Another aspect provides a method for enhancing anticancer immunity, including administering a polypeptide consisting of the amino acid sequence of SEQ ID NO: 1 to a subject in need thereof.

The terms “polypetide,” “subject,” “administration,” “cancer” and the like may be within the above-described ranges.

### Advantageous Effects of Disclosure

A pharmaceutical composition for the prevention or treatment of cancer or anticancer immune vaccine composition including a polypeptide consisting of the amino acid sequence of SEQ ID NO: 1, according to one aspect, can enhance anticancer immunity by activating dendritic cells and T cells. Furthermore, the composition can exhibit a remarkably excellent synergistic anticancer immune effect through administration in combination with an immune checkpoint inhibitor.

### BRIEF DESCRIPTION OF DRAWINGS

FIG. 1 is a graph showing the expression of TNF-α cytokine secreted in a concentration-dependent manner by poly6 stimulation in mouse-derived dendritic cells (statistical significance was tested by Student's t-test. *, P<0.05; **, P<0.01; ***, P<0.001; and ****, P<0.0001).

FIG. 2 illustrates the results of confirming the increase in NOS2 and nitric oxide in a type 1 interferon-dependent manner in dendritic cells by poly6 treatment, in which, specifically, FIG. 2(A) illustrates the results of confirming the increase in NOS2 through Western blot assay, after mouse-derived dendritic cells were treated with poly6 for 24 hr, and FIG. 2(B) illustrates the results of confirming through Nitrite/Nitrate kit assay that the nitrate level was increased in WT mice in a concentration-dependent manner, and was not increased in IFN K.O mice (statistical significance was tested by Student's t-test. *, P<0.05; **, P<0.01; and ***, P<0.001).

FIG. 3 is a graph showing the results of confirming the ability to differentiate into TNF-α/iNOS-producing DCs from bone marrow-derived dendritic cells (statistical significance was tested by Student's t-test. *, P<0.05; **, P<0.01; and ***, P<0.001).

FIG. 4 illustrates the expression patterns of molecular markers involved in maturation, by poly6 treatment in mouse-derived dendritic cells (statistical significance was tested by Student's t-test. *, P<0.05; **, P<0.01; ***, P<0.001; and ****, P<0.0001).

FIG. 5 is a graph showing the expression patterns of molecular markers involved in maturation by poly6 treatment in DC2.4 cells (statistical significance was tested by Student's t-test. *, P<0.05; **, P<0.01; and ***, P<0.001).

FIG. 6 illustrates the results of confirming the cytotoxicity of various cancer cell lines (MC38 murine colon cancer cell, B16F10 murine melanoma cancer cell, E0771 murine breast cancer cell, PanO2 murine pancreatic cancer cell, and MDA231 human breast cancer cell) by DC2.4 cells treated with poly6 at different concentrations, through FACS analysis (statistical significance was tested by Student's t-test and one-way-ANOVA. *, P<0.05; **, P<0.01; ***, P<0.001; and ****, P<0.0001).

FIG. 7 illustrates the results of confirming that the cytotoxicity of various cancer cell lines (MC38, B16F10, and EO771) was reduced when iNOS formation was inhibited by L-NAME (5 mM) treatment while stimulating DC2.4 cells with poly6 for 24 hours (statistical significance was tested by Student's t-test. *, P<0.05; **, P<0.01; and ***, P<0.001).

FIG. 8 illustrates the results of confirming that 3-nitrotyrosine was accumulated in cancer cells when MC38 cancer cells were treated with a culture medium in which nitric oxide had been accumulated, for 4 hours after DC2.4 cells were stimulated with poly6 for 48 hours (statistical significance was tested by Student's t-test. *, P<0.05; **, P<0.01; and ***, P<0.001).

FIG. 9 illustrates the results confirmed through confocal microscopy after DC2.4 cells were stimulated with poly6 for 48 hours, and MC38 cancer cells were treated with a culture medium in which nitric oxide had been accumulated, for 4 hours, followed by fixation and permeabilization of the cancer cells and staining with a 3-nitrotyrosine antibody for 30 minutes, and illustrates the results of analyzing fluorescence mean intensity through lmagej program, through which it was confirmed that the 3-nitrotyrosine level was statistically significantly increased in MC38 cancer cells by the culture medium of poly6-treated dendritic cells (statistical significance was tested by Student's t-test. *, P<0.05; **, P<0.01; and ***, P<0.001).

FIG. 10 illustrates the results of confirming the degree of accumulation of 3-nitrotyrosine in tumor tissue, the results of evaluation using a confocal microscopy 63× lens, and the results of measuring fluorescence mean intensity by using lmagej program (statistical significance was tested by Student's t-test. *, P<0.05; **, P<0.01; and ***, P<0.001).

FIG. 11 illustrates the results of confirming an anticancer effect in C57BL/6 mice into which MC38 cancer cells were injected, in which, specifically, (A) illustrates an in vivo animal experiment schedule, (B) illustrates the results of confirming the tumor growth rate through tumor size measurement, (C) is an image illustrating tumors extracted on day 16 after cancer cell injection, and (D) is a graph showing the results of measuring tumor weight on day 16 (statistical significance was tested by Student's t-test. *, P<0.05; **, P<0.01; and ***, P<0.001).

FIG. 12 illustrates the results of confirming an anticancer effect in melanoma B16F10, in which, specifically, (A) illustrates an in vivo animal experiment schedule, (B) illustrates the results of confirming the tumor growth rate through tumor size measurement, (C) is an image illustrating tumors extracted on day 12 after cancer cell injection, and (D) is a graph showing the results of measuring tumor weight on day 12 (statistical significance was tested by Student's t-test. *, P<0.05; **, P<0.01; and ***, P<0.001).

FIG. 13 illustrates the Hematoxylin and eosin staining results of MC38 colon cancer tissue.

FIG. 14 illustrates the results of confirming an anticancer effect in C57BL/6 mice into which MC38 cancer cells were injected, in which, specifically, (A) illustrates an in vivo animal experiment schedule, (B) illustrates the results of confirming the tumor growth rate through tumor size measurement, (C) is an image illustrating tumors extracted on day 23 after cancer cell injection, and (D) is a graph showing the results of measuring tumor weight on day 23 (statistical significance was tested by Student's t-test. *, P<0.05; **, P<0.01; and ***, P<0.001).

FIG. 15 illustrates the results of confirming the apoptotic cell death of C57BL/6 mice into which MC38 cancer cells were injected, through TUNEL assay (statistical significance was tested by Student's t-test. *, P<0.05; **, P<0.01; and ***, P<0.001).

FIG. 16 illustrates the results of confirming CD8 T cell-mediated CTL response in MC38 tumor tissue (statistical significance was tested by Student's t-test. *, P<0.05; **, P<0.01; and ***, P<0.001).

FIG. 17 illustrates the results of confirming effector T cells by using a flow cytometer, in which, specifically, tumor tissue was dissociated and separated into single cells, and surface T cell markers were stained with CD3, CD4, or CD8, followed by fixation and permeabilization, staining with a TNF- or IFN-γantibody through an intracellular cytokine staining method, and analysis using a flow cytometer (statistical significance was tested by Student's t-test. *, P<0.05; **, P<0.01; and ***, P<0.001)

FIG. 18 illustrates the results of analyzing T cells in MC38 tumor tissue through a flow cytometer, in which, specifically, (A) illustrates the results of confirming the increase in CD4 and CD8 T cells in tumor tissue according to poly6 injection, and (B) illustrates the results of confirming the increase in CD44/CD25-activated CD4/CD8 T cells in tumor tissues of poly6 groups (statistical significance was tested by Student's t-test. *, P<0.05; **, P<0.01; and ***, P<0.001).

FIG. 19 illustrates NK cell population, in which, specifically, the population of NK cells was confirmed through FACS analysis (statistical significance was tested by Student's t-test. *, P<0.05; **, P<0.01; and ***, P<0.001).

FIG. 20 illustrates the results of analyzing activated T cells in B16F10 tumor tissue by using a flow cytometer (statistical significance was tested by Student's t-test. *, P<0.05; **, P<0.01; and ***, P<0.001).

FIG. 21 illustrates the results of confirming increased dendritic cells in tumor tissue and spleen cells, in which, specifically, tumors and spleens were separated and dissociated, and CD11b- and CD11c-positive cells were confirmed using a flow cytometer (statistical significance was tested by Student's t-test. *, P<0.05; **, P<0.01; and ***, P<0.001).

FIG. 22 illustrates the results of confirming Tip-DC population in MC38 tumor tissue and spleen, in which, specifically, the induction of Tip-DCs was confirmed in poly6-stimulated groups by flow cytometry (*, P<0.05; **, P<0.01; and ***, P<0.001.).

FIG. 23 illustrates the results of confirming Tip-DC population in B16F10 tumor tissue.

FIG. 24 illustrates the results of confirming the degree of maturation through maturation markers of dendritic cells in tumor tissues and lymph nodes, in which, specifically, tumor tissues and lymph nodes were dissociated and separated into single cells, and then dendritic cells were stained with maturation markers, and the degree of maturation of dendritic cells was evaluated by flow cytometry (*, P<0.05; **, P<0.01; and ***, P<0.001.).

FIG. 25 is a graph confirming the number of macrophages in MC38 tumors by flow cytometry (*, P<0.05; **, P<0.01; and ***, P<0.001).

FIG. 26 illustrates graph confirming an anticancer effect by poly6 in a model in which HBV W4P large surface proteins-expressing NIH-3T3 cells (1×10⁸) were injected into balb nu/nu mice, in which reduced tumor size and reduced tumor weight were confirmed (*, P<0.05; **, P<0.01; and ***, P<0.001).

FIG. 27 illustrates the results of confirming anticancer effects when anti PD-L1 as an immune checkpoint inhibitor and poly6 as an adjuvant were used in combination, in which, specifically, (A) illustrates an in vivo animal experiment schedule, (B) is a graph confirming the tumor growth rate through tumor size measurement, (C) is an image showing tumors extracted on day 21 after cancer cell injection, and (D) is a graph showing the results of measuring tumor weight on day 21 (statistical significance was tested by Student's t-test. *, P<0.05; **, P<0.01; and ***, P<0.001).

### MODE OF DISCLOSURE

Hereinafter, the present disclosure will be described in further detail with reference to the following examples. However, these examples are provided for illustrative purposes and are not intended to limit the scope of the present disclosure.

### Example

### 1. Differentiation into TNF-α/NOS2-producing Dendritic Cells by Poly6 in Type 1 Interferon-Dependent Manner

### (1) Measurement of TNF-α Cytokine Expression

Bone marrow-derived dendritic cells were obtained from C57BL/6 mice and interferon knockout mice.

Mouse-derived dendritic cells were treated with poly6 peptide (GRLVFQ, SEQ ID NO: 1) at different concentrations (10 pM, 1 nM, 100 nM, and 10 μM), and then TNF-α cytokine secreted by the dendritic cells was measured by ELISA.

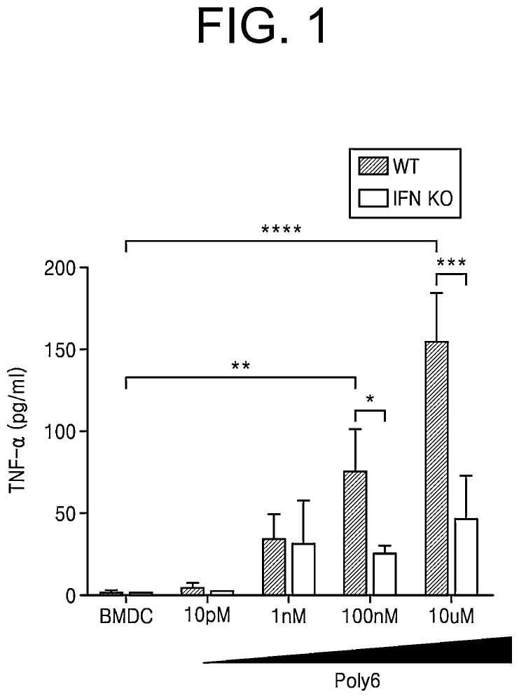

As a result, it was confirmed that the expression of TNF-α cytokine was increased by poly6 treatment in a concentration-dependent manner. It was also confirmed that, compared to C57BL/6 mice, the amount of secreted TNF-α was statistically significantly reduced in interferon knockout mice when treated with 100 nM or 10 μM of poly6 (FIG. 1).

### (2) Confirmation of Increase in NOS2 and Nitric Oxide

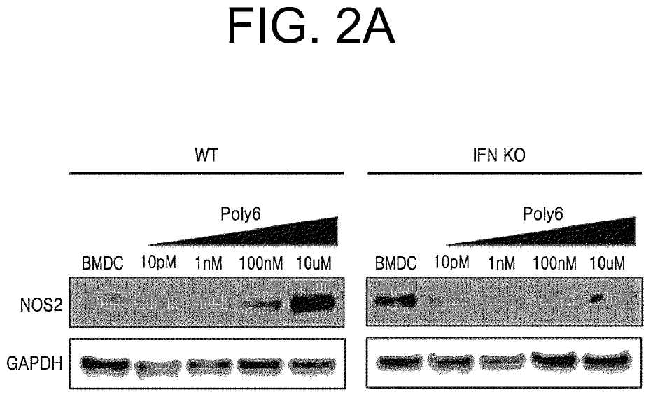

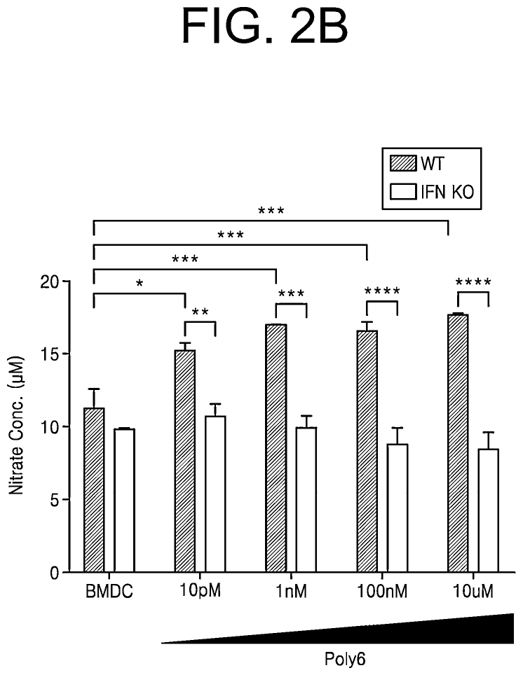

Mouse-derived dendritic cells were treated with poly6 peptide at different concentrations, the protein was obtained by prep using RIPA lysis buffer from cell pellets, and it was confirmed through Western blotting assay that NOS2 increased in a concentration-dependent manner in WT C57BL/6 mice, whereas it was confirmed that NOS2 did not increase in IFN K.O mice, but rather decreased (FIG. 2A).

It was confirmed using a nitrite/nitrate kit that nitrate concentration in a cell medium was increased by poly6 treatment, and was not increased in IFN K.O mice (FIG. 2B).

Through these results, it was confirmed that dendritic cells increased TNF-α and iNOS by poly6 treatment in a type 1 interferon-dependent manner.

### (3) Confirmation of Ability to Induce Differentiation into TNF-α/iNOS-Producing Dendritic Cells

Since it was confirmed that TNF-α and iNOS were increased in a type 1 interferon-dependent manner, the ability to induce differentiation into NF-α/iNOS-producing dendritic cells from bone marrow-derived dendritic cells was examined.

Bone marrow-derived dendritic cells were stimulated with poly6 for 24 hr, followed by fixation with 1% paraformaldehyde and permeabilization with 0.1% Triton X-100, and TNF-α/iNOS-producing dendritic cells were analyzed using a flow cytometer.

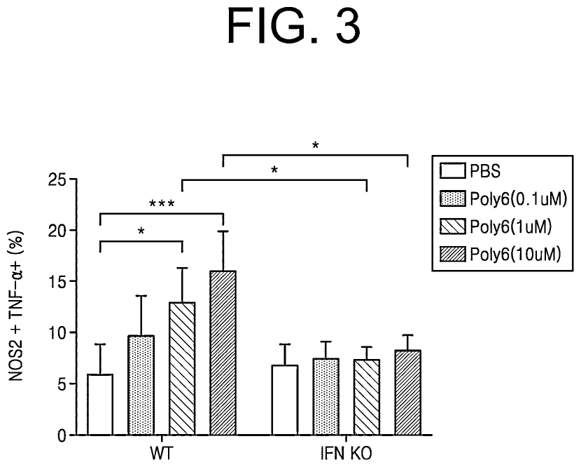

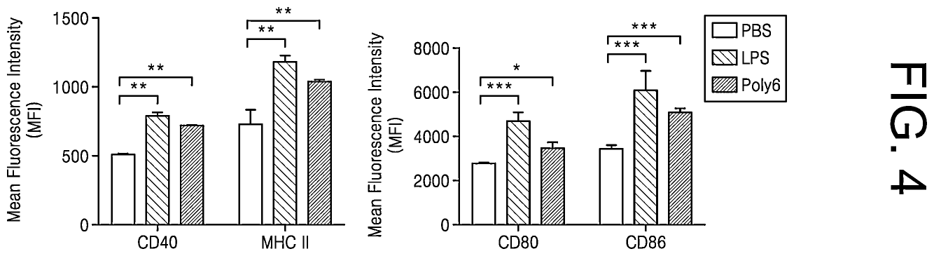

As a result, it was confirmed that Tip-DCs (TNF-α/iNOS-producing dendritic cells) were formed in bone marrow-derived dendritic cells of WT mice by poly6 treatment in a concentration-dependent manner. In contrast, it was confirmed that Tip-DCs did not differentiate in IFN K.O mice (FIG. 3).

### 2. Activation of Dendritic Cells by Poly6

### (1) Measurement of Expression of CD40, CD80, CD86, and MHCII

Mouse-derived dendritic cells were treated with poly6 peptide (10 μM) for 24 hours, and then, the expression levels of CD40, CD80, CD86, and MHCII, which are molecular markers involved in the maturation of dendritic cells, were measured using a flow cytometer.

As a result, it was confirmed that the expression of CD40, CD80, CD86 and MHCII molecules in dendritic cells was increased by poly6 treatment (FIG. 4).

### (2) Confirmation of Dendritic Cell Activation in DC2.4 Cells

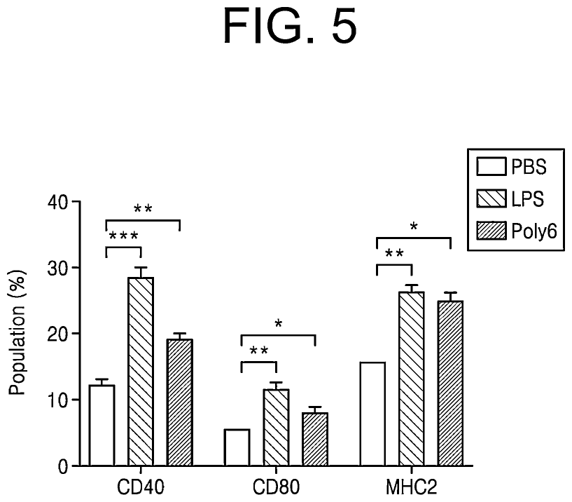

A DC2.4 cell line was starved for 30 minutes, and cultured along with poly6 in a medium supplemented with 1% FBS, 100 U/mL penicillin, and 100 μg/mL streptomycin for 24 hours, and then dendritic cells in which CD40, CD80 and MHCII molecules involved in the maturation of dendritic cells were expressed were identified by flow cytometry (FIG. 5).

### 3. Nitric Oxide (NO)-Dependent Anticancer Effect of Poly6

### (1) Cytotoxicity against Various Cancer Cell Lines by DC2.4 Cells Treated with Poly6 at Different Concentrations

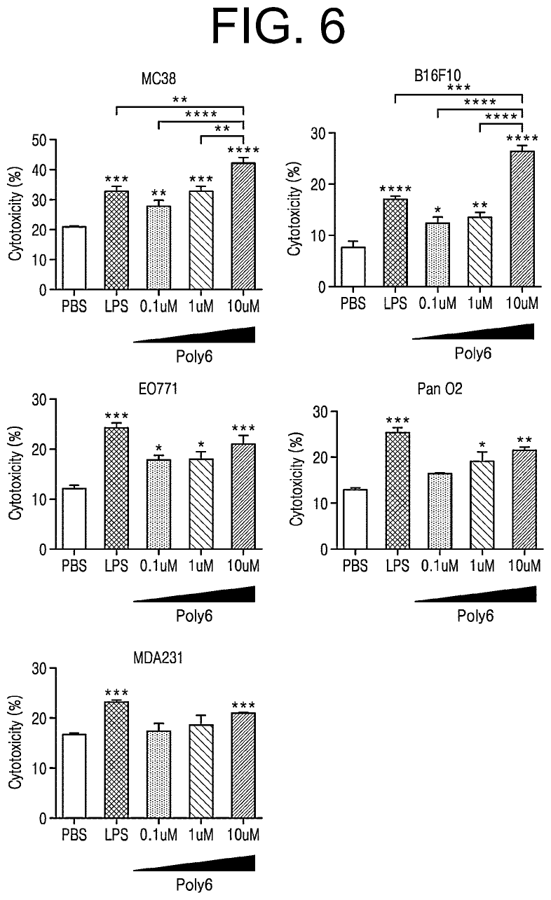

Direct cytotoxicity was induced in several cancer cell lines by poly6-stimulated DC2.4 cells. It was confirmed that DC2.4 was stimulated by poly6 treatment in a concentration-dependent manner and cytotoxicity was induced in cancer cell lines (MC38, B16F10, EO771, PanO2, and MDA231). In particular, it was confirmed that, for the MC38 and B16F10 cancer cell lines, the cytotoxicity against cancer cells was statistically significantly increased in DC2.4 cells treated with 10 μM of poly6 compared to when treated with LPS (1 μg/ml) (FIG. 6).

### (2) When iNOS Formation was Inhibited

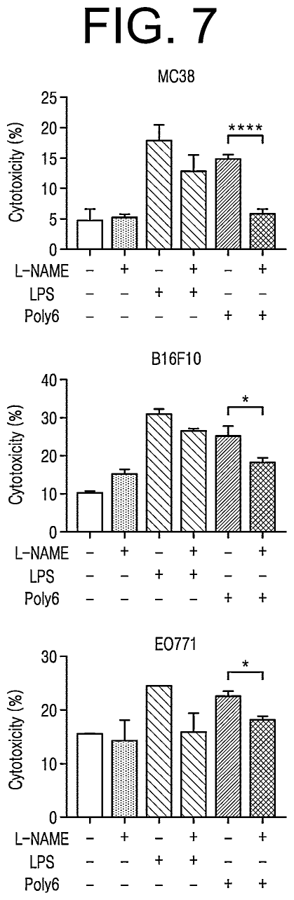

It was also confirmed that cytotoxicity was reduced when poly6-treated DC2.4 cells and cancer cells were co-cultured, due to iNOS inhibition by L-NAME (FIG. 7).

### (3) Whether 3-nitrotyrosine was Accumulated in Cancer Cells

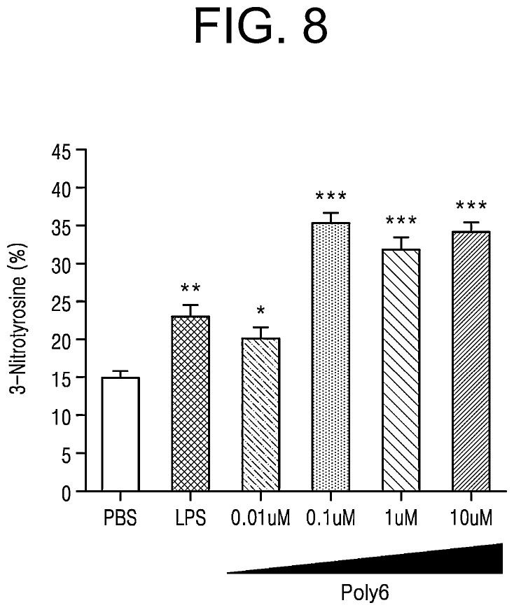

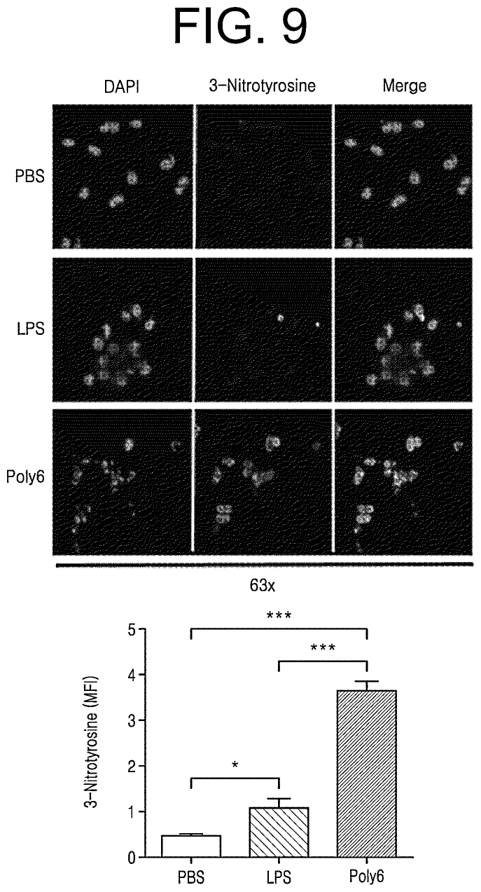

When cancer cells were treated with DC2.4 cells stimulated with poly6 for 48 hours in a culture medium in which nitric oxide was accumulated, the increase in the level of 3-nitrotyrosine, which is an indicator of peroxynitrite, was confirmed by flow cytometry (FIG. 8). It was also confirmed through Immunofluorescence (FIG. 9).

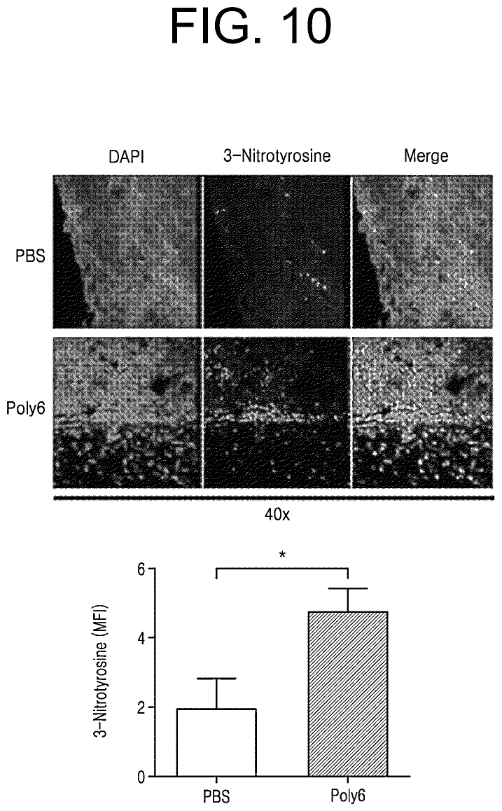

Furthermore, the increase of 3-nitrotyrosine in tumor tissue was also confirmed using confocal microscopy through an immunofluorescence assay method (FIG. 10).

### 4. Function of Poly6 as Anticancer Vaccine

### (1) Confirmation of Efficacy in Colcon Cancer

MC38 colon cancer cells (1×10⁶ cells) were injected subcutaneously into C57BL/6 mice to form tumors, and poly6 peptide (10 μg) was injected at a location away from the cancer cell injection site to confirm an anticancer immune effect.

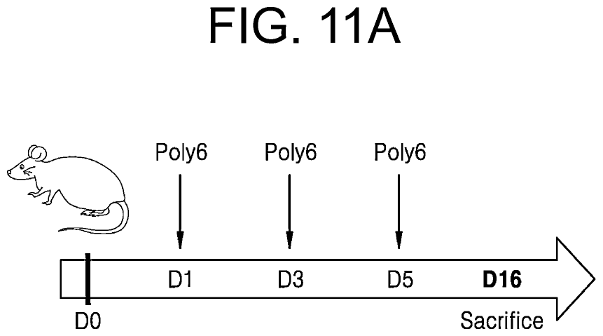

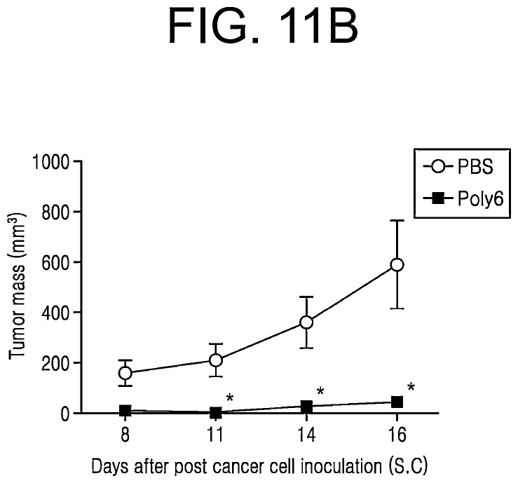

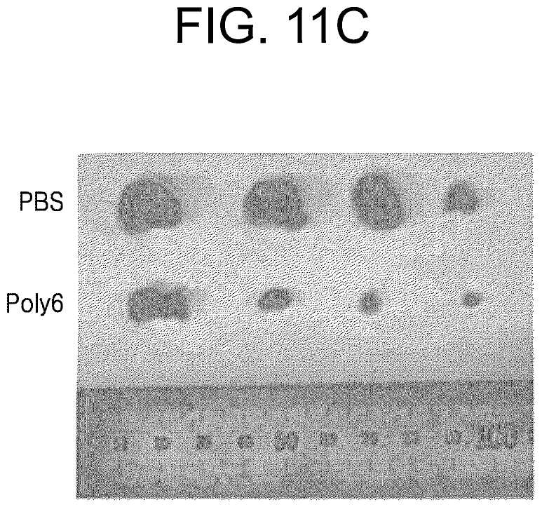

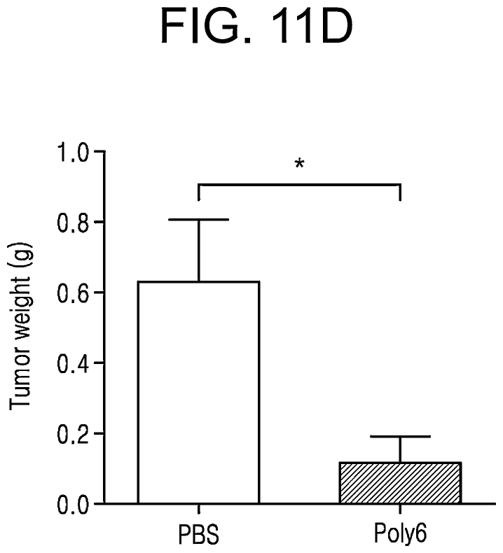

As a result, t was confirmed that, compared to a PBS control, the tumor growth rate was reduced, and finally, tumor mass and tumor weight were reduced (FIG. 11).

### (2) Confirmation of Efficacy in Melanoma

In addition, B16F10 melanoma cancer cells (1×10⁶ cells) were injected subcutaneously into C57BL/6 mice to form tumors, and poly6 peptide (10 μg) was injected at a location away from the cancer cell injection site to confirm an anticancer immune effect.

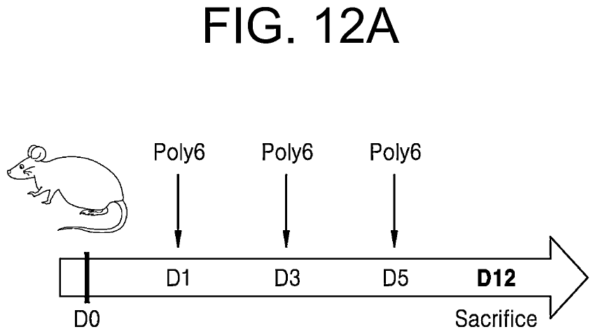

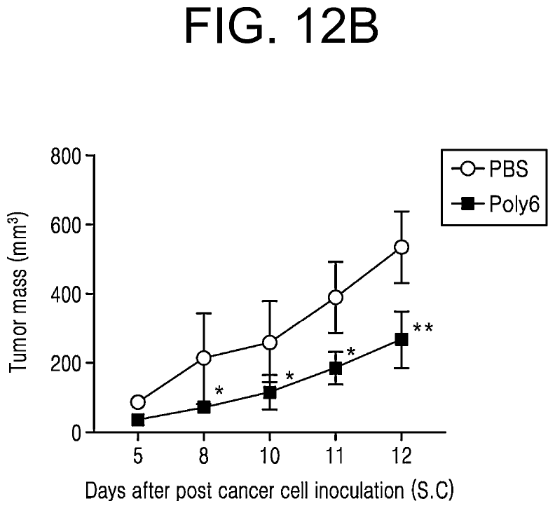

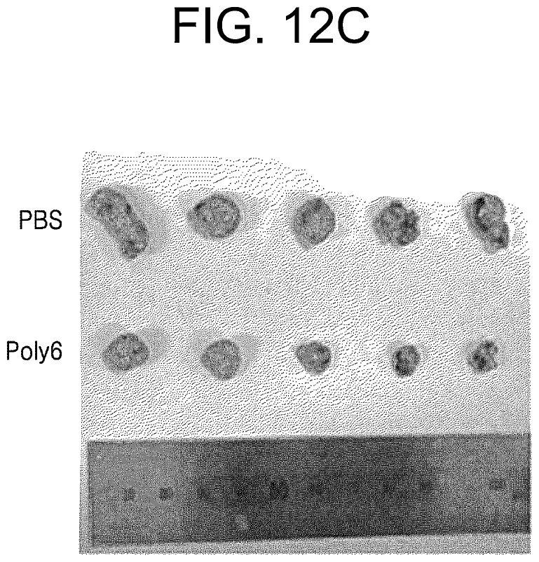

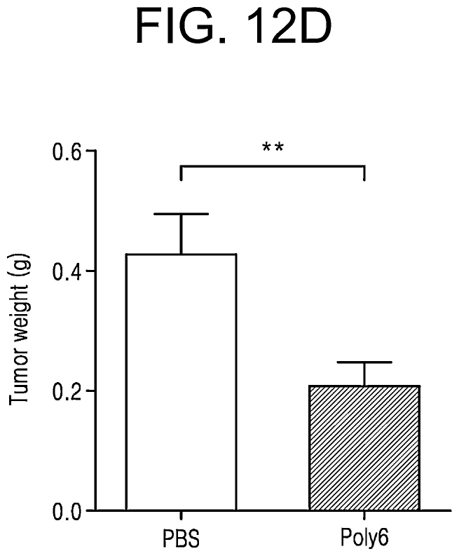

As a result, it was confirmed that, compared to a PBS control, the tumor growth rate was reduced, and finally, tumor mass and tumor weight were reduced (FIG. 12).

### (3) Histological Staining Results

Tumors were extracted from MC38-bearing mice, fixed with 4% paraformaldehyde, and then subjected to hematoxylin and eosin staining through paraffin section.

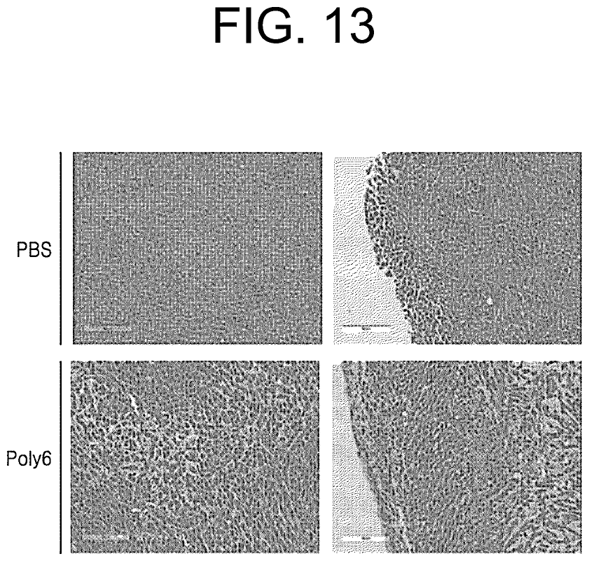

As a result, it was confirmed that density in tumor tissue was reduced in a poly6-treated group, compared to a PBS control. Additionally, it was confirmed that immune cells gathered at the edge of tumors in the poly6-treated group (FIG. 13).

### (4) Confirmation of Anticancer Effect according to Injection of Poly6 Peptide Before Injection of MC38 Cancer Cells

Poly6 peptide (10 μg) was additionally injected one day before cancer cell injection to induce immune enhancement as an anticancer immune vaccine in the mouse body, and then poly6 was additionally injected three times to confirm a long-term anticancer effect up to day 23.

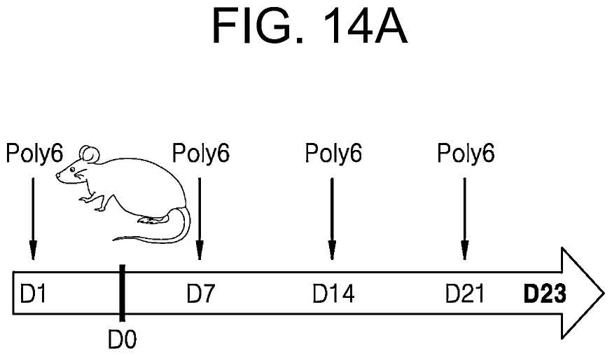

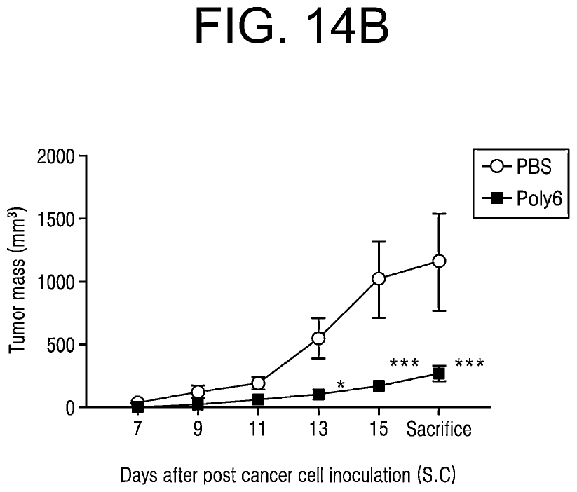

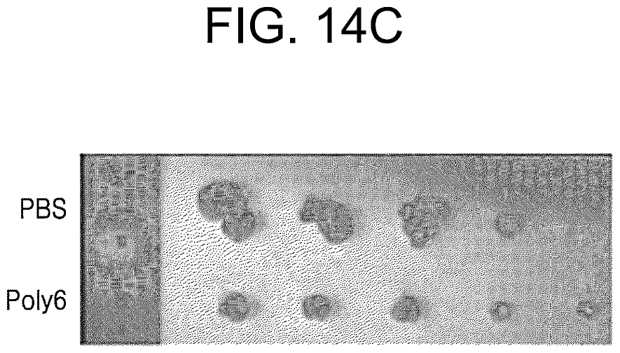

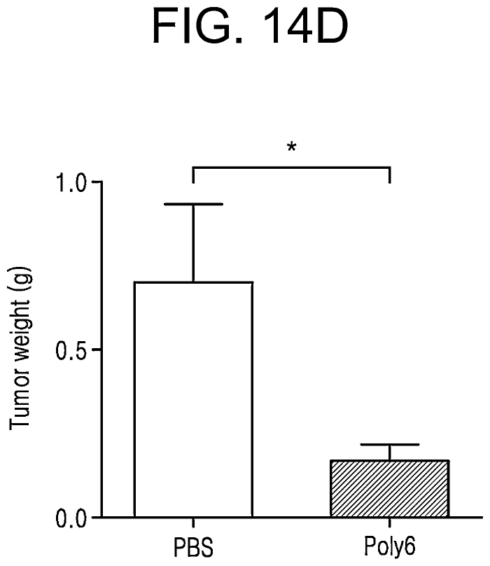

As a result, it was confirmed that, compared to a PBS control, the tumor growth rate was reduced, and finally, tumor mass and tumor weight were reduced (FIG. 14).

### (5) TUNEL Assay

The terminal deoxynucleotidyl transferase-mediated dUTP nick-end labeling (TUNEL) assay was carried out using the ApopTag Peroxidase In Situ Apoptosis Detection Kit (Millipore).

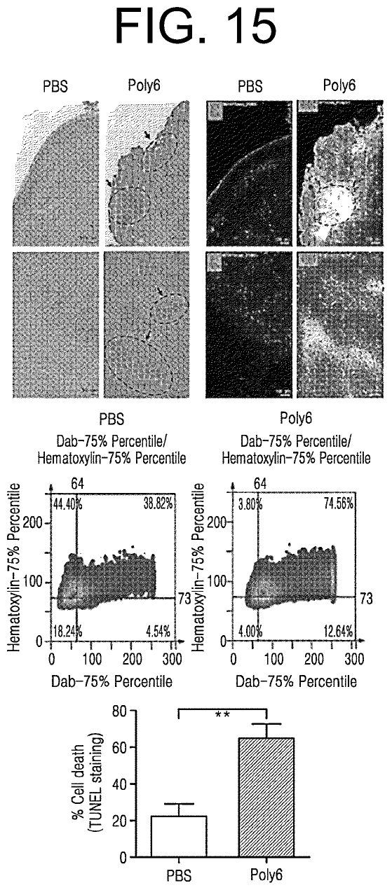

As a result, greatly increased apoptotic cell death was confirmed in MC38 tumor tissue compared to a PBS control. Positive staining was observed not only at the edge of tissue but also inside the tissue. This was digitized through the HistoQuest (TissueGnostics) program of Tissue FAXS to analyze the Dab positive cell population (FIG. 15).

### (6) Confirmation of CD8 T Cell-Mediated CTL Response

The mRNA levels of cell lytic proteins (granzymeB and perforin), pro-apoptotic proteins (Bax, bak, and cytochrome C), and death signal inducing ligands (TRAIL, Fas, and Fas L) were identified in MC38 tumor tissues.

Specifically, tumor tissue was sectioned, and total mRNA was prepped through the Trizol method, and analyzed by qRT-PCR using each primer set.

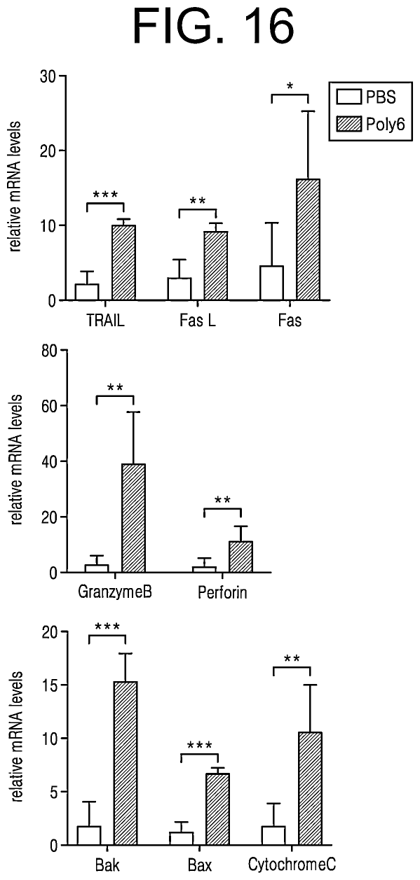

As a result, it was confirmed that the mRNA levels of cytolytic proteins, pro-apoptotic proteins, and death signal ligands were statistically significantly increased in the poly6-stimulated tumor tissue (FIG. 16).

### 5. Anticancer Immune Action by T Cell Activation and Tip-DC Formation in Mice In Vivo

### (1) Confirmation of T Cell Activity

### 1) Identification of Effector T Cells

MC38 colon cancer cells (1×10⁶ cells) were injected subcutaneously into

C57BL/6 mice to form tumors, separated into single cells through tumor dissociation, and then TNF-α/IFN-α-producing CD4 and CD8 T cells were identified by flow cytometry.

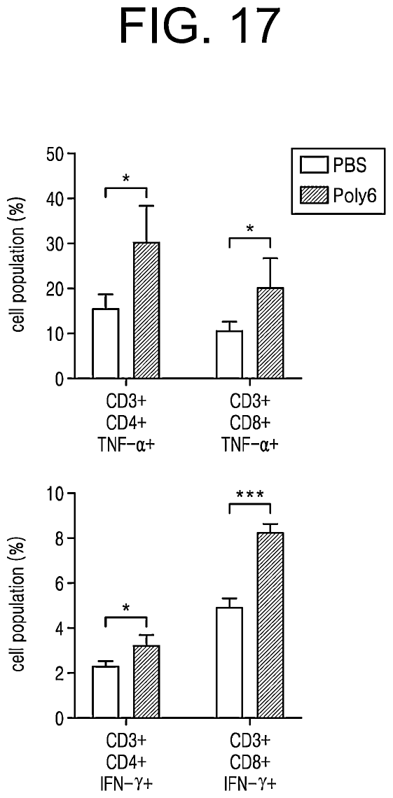

As a result, it was confirmed that both CD4 and CD8 T cells producing TNF-α or IFN-γ, which function as effectors, were increased in poly6-stimulated tumor tissue (FIG. 17).

### 2) T Cell Analysis in MC38 Tumor Tissue

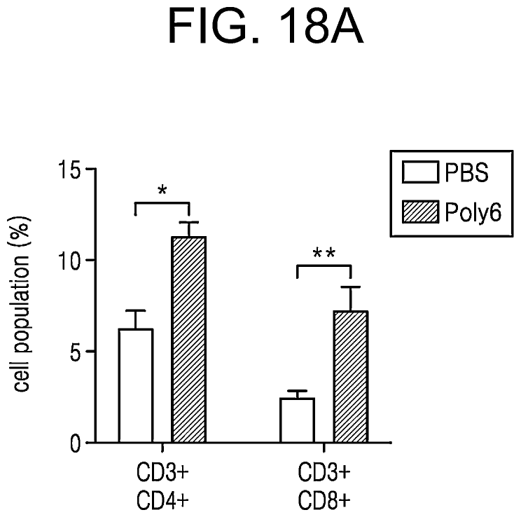

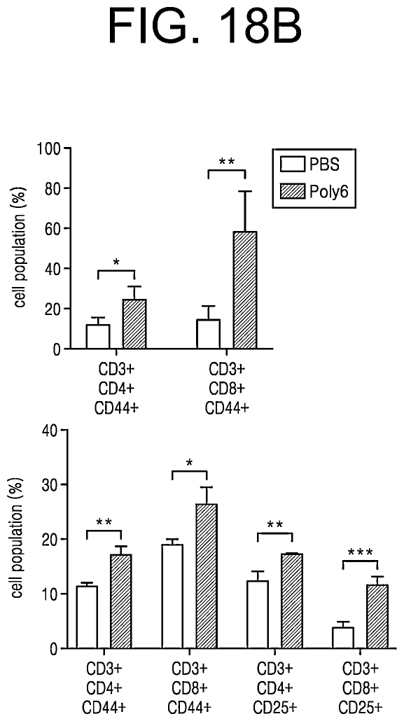

It was also confirmed that, compared to a control, the number of T cells increased in poly6-stimulated tumor tissue, and CD44- and CD25-positive cells, which are markers of activated T cells, were increased (FIG. 18).

### 3) Identification of NK Cells

In contrast, natural killer cells were increased in tumor tissue and spleen by poly6 stimulation, but statistically significant results were not obtained (FIG. 19).

4) Confirmation of Increase in TNF-α+, CD4+ and TNF-α+, CD8+ T Cells in Melanoma

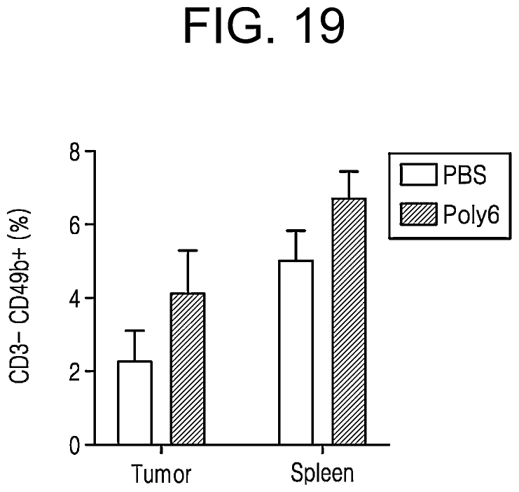

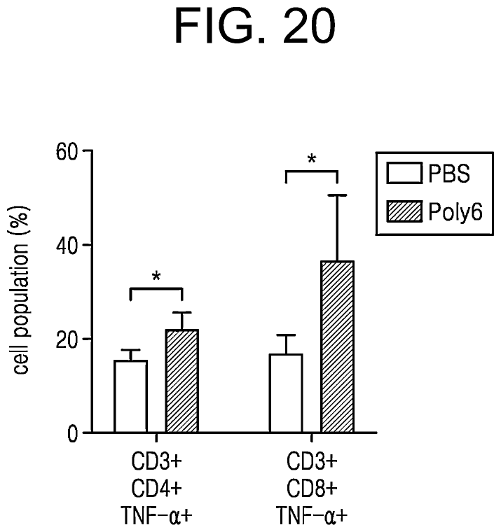

Additionally, it was confirmed that TNF-α+ CD4+ and TNF-α+ CD8+ T cells were increased in tumor tissue in B16F10 melanoma carcinoma, in addition to MC38 colon cancer (FIG. 20).

Through this result, it was confirmed that the ability to induce T cell activity by poly6 was not specific to colon carcinoma, but showed the ability to non-specifically induce immunity against various carcinomas.

### (2) Confirmation of Tip-DC Formation

### 1) Identification of Number of Dendritic Cells

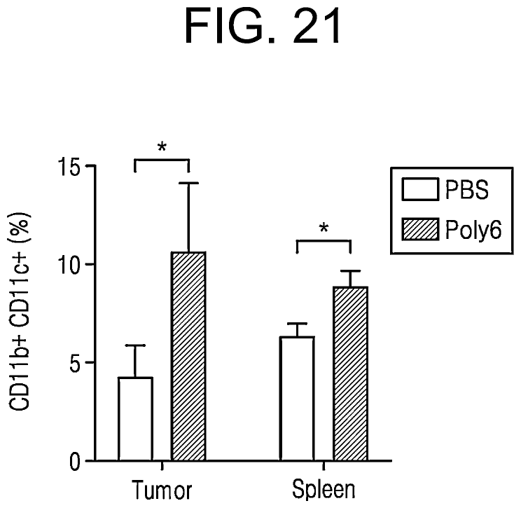

Specifically, it was confirmed that the number of dendritic cells statistically significantly increased in MC38 tumor tissue and spleen in a poly6-injected group (FIG. 21).

### 2) Confirmation of Ability to Induce Tip-DCs

Since it was confirmed that poly6 exhibited the ability to induce Tip-DCs in dendritic cells in vitro, the characteristics of increased dendritic cells were examined.

CD11b+, CD11c+, MHC2+, TNF-α+, and NOS2+ cells were analyzed in vivo using dendritic cell intracellular cytokine staining to confirm and evaluate the ability to induce differentiation into Tip-DCs.

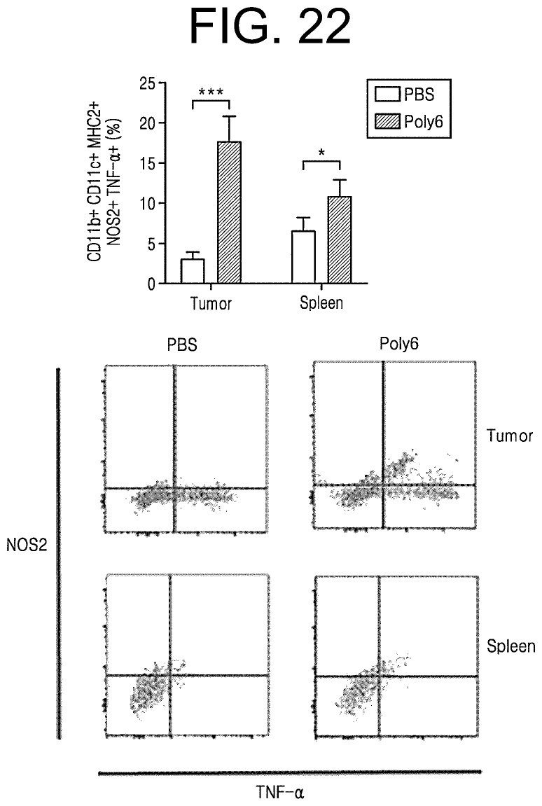

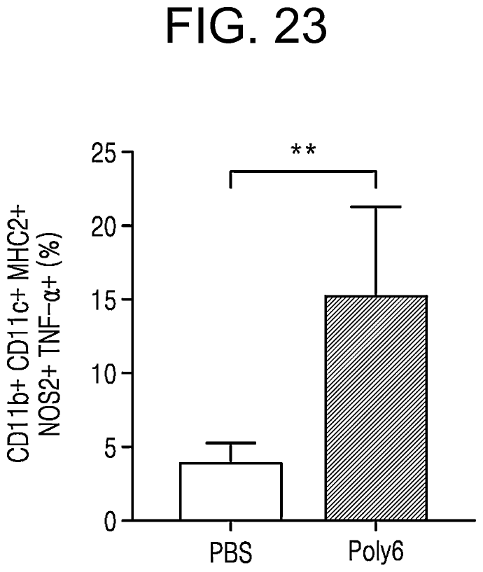

As a result, statistically significant formation of the Tip-DC population was confirmed in both MC38 tumor tissue and spleen in a poly6-stimulated group (FIG. 22).

### 3) Confirmation of Tip-DC Induction Ability in Melanoma

Additionally, it was confirmed that Tip-DCs were increased in tumor tissue in B16F10 melanoma carcinoma, in addition to MC38 colon cancer (FIG. 23).

Through this result, it was confirmed that the ability to induce Tip-DCs by poly6 was not specific to colon carcinoma, and the ability to non-specifically induce immunity against various carcinomas was shown.

### (3) Confirmation of Degree of Maturation of Dendritic Cells

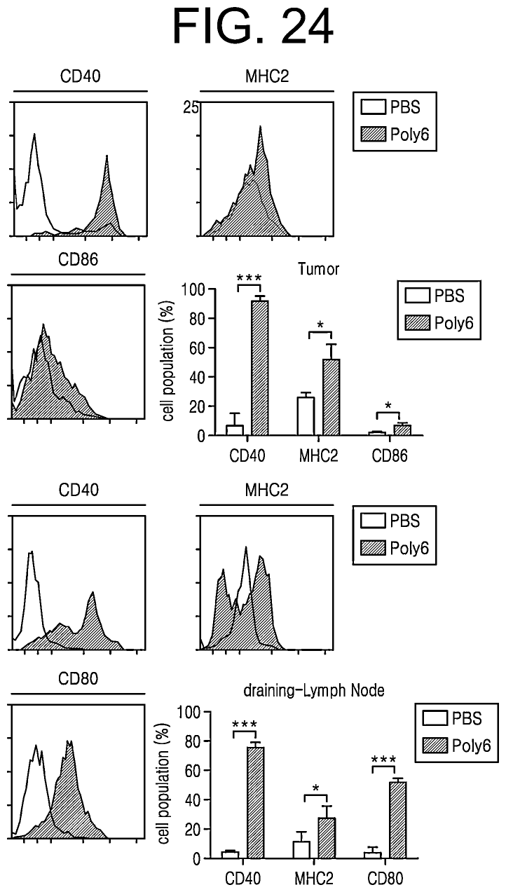

It was confirmed that the maturation of dendritic cells was enhanced in MC38 tumor tissue and draining lymph nodes. It was confirmed that CD40, MHCII, and CD86 were increased in the tumor tissue, and CD40, MHC2, and CD80 were increased in the draining lymph nodes. In particular, the expression of CD40 showed a drastic increase in both tumor tissue and lymph nodes (FIG. 24).

It was expected to show the ability to induce T cell activation using the CD40/CD40L axis.

### (4) Case of Macrophages

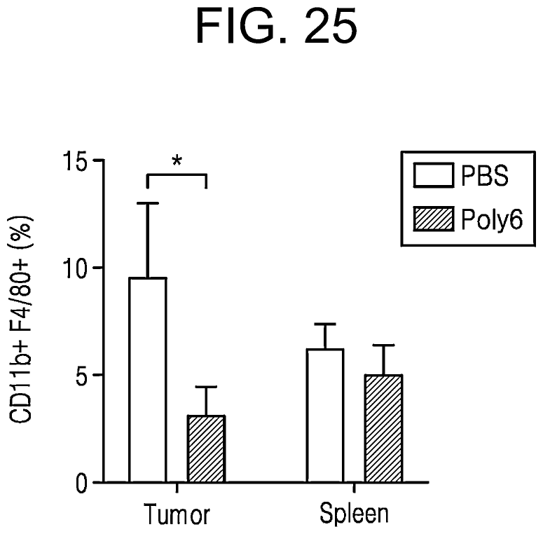

Among the innate immune cells in MC38 tumors, the number of macrophages statistically significantly decreased in tumor tissue and decreased in the spleen, but the results were not statistically significant (FIG. 25).

Taken together, these results indicate that poly6 induces T cell activity in vivo, increases the CTL response, and induces direct anticancer activity through the induction of differentiation into Tip-DCs.

### (5) In Absence of T Cells

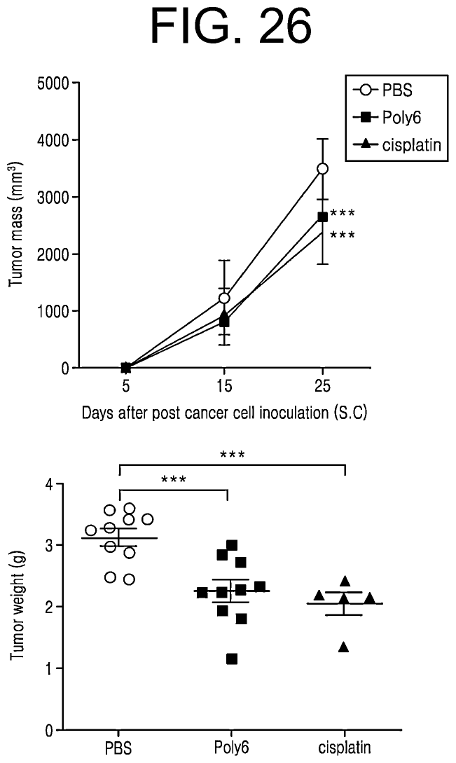

Additionally, the anticancer effect was also confirmed in balb/c nu/nu mice excluding T cells (FIG. 26), and this result supports the ability of Tip-DCs to directly induce anticancer activity, in that the anticancer effect is induced by the innate immune cells.

### 6. Induction of Enhanced Anticancer Immunity by Poly6 as Adjuvant when Treated in Combination with anti PD-L1 as Immune Checkpoint Inhibitor

Animal experiments were conducted to evaluate the enhanced ability to induce anticancer activity through the combined effect of poly6 and anti PD-L1, which is a commercially available immune checkpoint inhibitor.

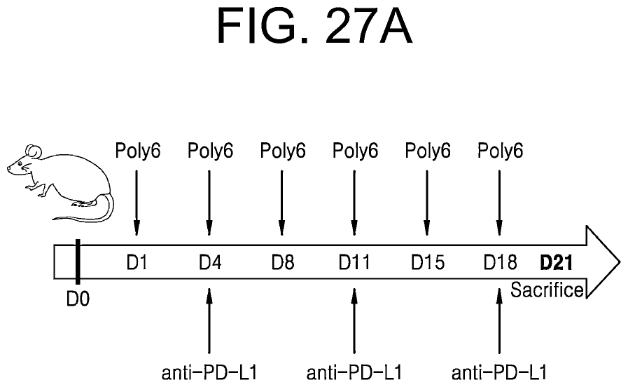

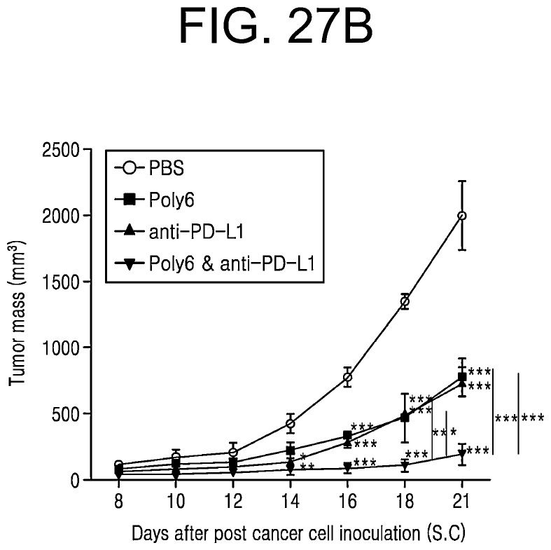

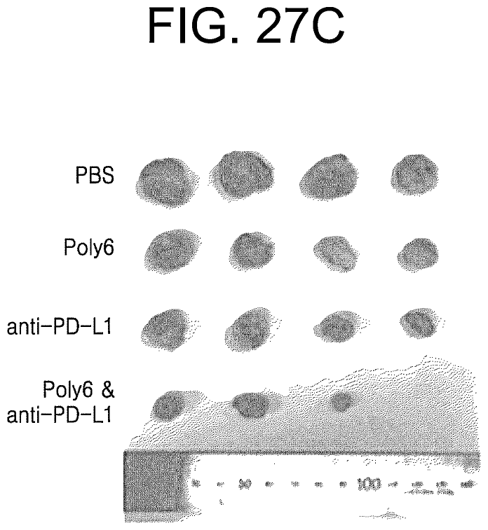

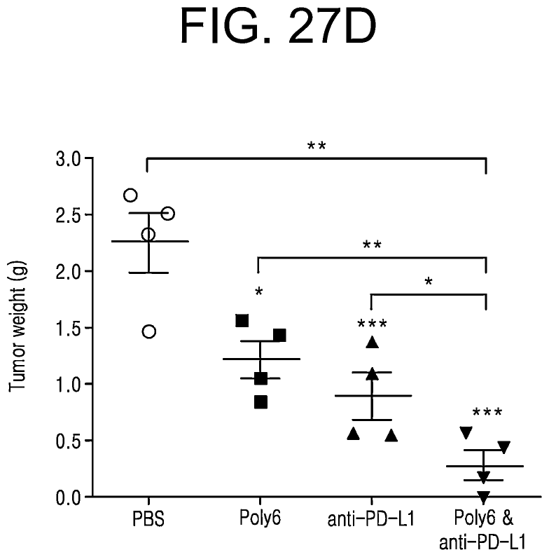

As a result, statistically significant enhanced anticancer activity was observed compared to a PBS control, an anti PD-L1 alone group, or a poly6 alone group. This was confirmed by measuring the tumor growth rate and tumor mass and weight on day 21 after cancer cell injection (FIG. 27).

1-20. (canceled)

21. A method of preventing or treating cancer, comprising administering a polypeptide comprising the amino acid sequence of SEQ ID NO: 1 to a subject in need thereof.

22. The method of claim 21, wherein the polypeptide activates dendritic cells and T cells.

23. The method of claim 21, wherein the polypeptide increases the expression of at least one selected from the group consisting of TNF-α and iNOS in dendritic cells.

24. The method of claim 21, wherein the cancer is at least one selected from the group consisting of colon cancer, melanoma, lung cancer, liver cancer, kidney cancer, gastric cancer, pancreatic cancer, rectal cancer, breast cancer, thyroid cancer, head and neck cancer, brain tumor, kidney cancer, bladder cancer, and prostate cancer.

25. The method of claim 21, further comprising administering an immune checkpoint inhibitor to the subject.

26. The method of claim 21, wherein the polypeptide is administered in combination with an immune checkpoint inhibitor.

27. The method of claim 25, wherein the immune checkpoint inhibitor is anti-PD-L1.

28. A method of enhancing anticancer immunity, comprising administering a polypeptide comprising the amino acid sequence of SEQ ID NO: 1 to a subject in need thereof.

29. The method of claim 28, wherein the polypeptide activates dendritic cells and T cells.

30. The method of claim 28, wherein the polypeptide increases the expression of at least one selected from the group consisting of TNF-α and iNOS in dendritic cells.

31. The method of claim 28, wherein the cancer is at least one selected from the group consisting of colon cancer, melanoma, lung cancer, liver cancer, kidney cancer, gastric cancer, pancreatic cancer, rectal cancer, breast cancer, thyroid cancer, head and neck cancer, brain tumor, kidney cancer, bladder cancer, and prostate cancer.

32. The method of claim 28, further comprising administering an immune checkpoint inhibitor to the subject.

33. The method of claim 28, wherein the polypeptide is administered in combination with an immune checkpoint inhibitor.

34. The method of claim 33, wherein the immune checkpoint inhibitor is anti-PD-L1.


SEQUENCE LISTING (1 sequence)

SEQ ID NO: 1 - protein, 6 aa, Artificial Sequence
  poly6 peptide
GRLVFQ